(19) United States
(12) Patent Application Publication
(10) Pub. No.: US 2020/0113500 A1
(43) Pub. Date: Apr. 16, 2020

(54) MENSTRUAL CUP HAVING BLOOD TEST CARTRIDGE

(71) Applicant: LOON LAB INC., Seongnam-si, Gyeonggi-do (KR)

(72) Inventor: Ryong HWANG, Cheonan-si, Chungcheongnam-do (KR)

(73) Assignee: LOON LAB INC., Seongnam-si, Gyeonggi-do (KR)

(21) Appl. No.: 16/604,206

(22) PCT Filed: Mar. 21, 2018
(86) PCT No.: PCT/KR2018/003315

§ 371 Date: Dec. 10, 2019

(30) Foreign Application Priority Data
Apr. 10, 2017 (KR) 10-2017-0045950
Mar. 6, 2018 (KR) 10-2018-0026234
Mar. 19, 2018 (KR) 10-2018-0031745

(51) Int. Cl. A61B 5/15; G01N 33/49; A61F 5/455

(52) U.S. Cl. CPC A61B 5/150045; G01N 33/68; A61F 5/4553; G01N 33/49

(57) ABSTRACT

The present invention relates to a menstrual cup having a blood test cartridge. A menstrual cup according to one embodiment of the present invention comprises a body part having an opening formed at one side of the body part, a storage part configured to receive menstrual blood therein, and a cartridge receiving part connected to the storage part at a side opposite the opening, and a cartridge for blood test removably mounted in the cartridge receiving part.

### FIELD OF THE INVENTION

The present invention relates to a menstrual cup having a blood test cartridge, and more particularly, to a menstrual cup having a blood test cartridge, which is capable of obtaining biometric information from menstrual blood and diagnosing a disease through the biometric information.

### BACKGROUND

During menstruation, in order to allow females to act more comfortable, menstrual blood leakage prevention tools, such as sanitary napkins (pads), menstrual cups, and tampons, are used to prevent menstrual blood from leaking out of a vagina.

Among the menstrual blood leak prevention tools, the menstrual cups are generally larger in capacity than the sanitary napkins or the tampons and have little leakage of menstrual blood. Further, unlike the sanitary napkins or the tampons, the menstrual cups are reusable and are economical and eco-friendly. Further, the menstrual cups are used in the form of a body insertion type. Even when the menstrual cups are worn, not only exercise and activity including swimming are to be free but also safety is proven for a long period of time.

Meanwhile, the menstrual blood is periodically discharged with the endometrium and may contain a lot of pieces of information such as genetic information and protein information like general blood. Therefore, if the menstrual blood, which is periodically discharged, is tested, biometric information can be checked and used for health management without a separate blood collection.

In case of the conventional menstrual cup, during removal of the menstrual cup, a menstrual volume and a color and a condition of the menstrual blood may be visually checked, however, obtaining biometric information such as genetic information and protein information contained in the menstrual blood and diagnosing a disease through the biometric information are impossible.

In order to test menstrual blood in the menstrual cup, installing a blood test cartridge in the menstrual cup may be considered. However, it is not practical to install and use the blood test cartridge in the menstrual cup having a small size, which is inserted into the vagina.

### SUMMARY OF THE INVENTION

An objective of the present invention is to solve the above-described problems. Further, the present invention is directed to providing a menstrual cup which has a blood test cartridge and which is capable of obtaining biometric information through menstrual blood and diagnosing a disease.

The present invention is also directed to providing a menstrual cup in which a cartridge for blood test is removably mounted and which is capable of not only easily checking a blood test results obtained from the cartridge, but also replacing the cartridge according to the need of a user.

One aspect of the present invention provides a menstrual cup having a blood test cartridge, which includes a body part having an opening formed at one side of the body part, a storage part configured to receive menstrual blood therein, and a cartridge receiving part connected to the storage part at a side opposite the opening, and a cartridge for blood test removably mounted in the cartridge receiving part.

The menstrual cup according to one embodiment of the present invention may further include a handle formed to protrude from the side opposite the opening of the body part.

According to one embodiment of the present invention, a hook configured to mount and fix the cartridge may be formed between the cartridge receiving part and the storage part. Further, a groove may be formed in the cartridge at a position corresponding to the hook such that the hook is hooked when the cartridge is received in the cartridge receiving part.

According to one embodiment of the present invention, the body part may include a first body part and a second body part which can be separated and coupled by interposing the cartridge receiving part therebetween. Further, a screw portion may be formed in each of the first body part and the second body part for separation and coupling. Furthermore, the handle may be formed to protrude from the second body part.

According to one embodiment of the present invention, a cut-out portion configured to connect the cartridge receiving part to the outside may be formed outward the cartridge receiving part, and the cartridge may be insertable into the cartridge receiving part through the cut-out portion.

According to one embodiment of the present invention, the body part may include an inner cup and an outer cup which can be coupled to and separated from each other, and when the inner cup is coupled to the outer cup, the cartridge receiving part may be formed between the inner cup and the outer cup. Further, the cartridge may be mounted in the inner cup when the inner cup is separated from the outer cup, and the cartridge may be located in the cartridge receiving part formed between the inner cup and the outer cup when the inner cup is coupled to the outer cup.

According to one embodiment of the present invention, the cartridge may perform genetic analysis or protein analysis of blood or detect a disease. Further, the cartridge may have a structure in which a plurality of sections for blood test are stacked.

According to one embodiment of the present invention, the cartridge may include a wireless communication module. Further, the cartridge may be configured to transmit a test result to a user terminal.

In accordance with the present invention, a test for menstrual blood is performed through a cartridge provided in a menstrual cup, it is possible to easily obtain biometric information such as genetic information and protein information of a menstrual cup user and diagnose a disease.

Further, a cartridge for blood test can be mounted in and removed from the menstrual cup such that a blood test result obtained from the cartridge can be easily checked through a cartridge reader. Further, the menstrual cup can be reused and only the cartridge can be replaced and used according to biometric information which a user wants to obtain.

### BRIEF DESCRIPTION OF THE DRAWINGS

FIG. 1 is a perspective view illustrating a menstrual cup according to one embodiment of the present invention.

FIG. 2 is a cross-sectional view illustrating a menstrual cup according to one embodiment of the present invention.

FIG. 3 is an exemplary diagram illustrating an internal structure of a cartridge for blood test provided in the menstrual cup according to one embodiment of the present invention.

FIGS. 4A to 4C are diagrams illustrating a case in which a cartridge is mounted in a menstrual cup according to a first embodiment of the present invention.

FIGS. 5A to 5C are diagrams illustrating a case in which a cartridge is mounted in a menstrual cup according to a first embodiment of the present invention.

FIGS. 6A and 6B are diagrams illustrating a case in which a cartridge is mounted in a menstrual cup according to a second embodiment of the present invention.

FIGS. 7A and 7B are diagrams illustrating a case in which a cartridge is mounted in a menstrual cup according to a second embodiment of the present invention.

FIGS. 8A to 8C are diagrams illustrating a case in which a cartridge is mounted in a menstrual cup according to a third embodiment of the present invention.

FIGS. 9A to 9C are diagrams illustrating a case in which a cartridge is mounted in a menstrual cup according to a third embodiment of the present invention.

FIGS. 10A to 10C are diagrams illustrating a case in which a cartridge is mounted in a menstrual cup according to a fourth embodiment of the present invention.

### DETAILED DESCRIPTION

Hereinafter, exemplary embodiments of the present disclosure will be fully described in a detail which is suitable for implementation by those skilled in the art to which the present disclosure pertains with reference to the accompanying drawings.

In order to clearly describe the present invention, a description of a portion not related to the present invention will be omitted, and throughout this disclosure, like reference numerals will be assigned to like components. Further, a size, a thickness, a position, and the like of each component shown in the drawings are arbitrarily illustrated for convenience of description, and thus the present invention is not necessarily limited to those shown in the drawings. That is, it should be noted that specific shapes, structures, and features described herein can be changed and implemented from one embodiment to another embodiment without departing from the spirit and scope of the present invention, and a position or an arrangement of each component can also be changed without departing from the spirit and scope of the present invention.

Accordingly, the following detailed description is not to be taken in a limiting sense, and the scope of the present invention should be construed to include the scope of the appended claims and equivalents thereof.

Meanwhile, in the following description, connecting two members or spaces is used to mean not only the two members or spaces are connected by being in direct contact with each other but also the two members or spaces are connected to each other through another member or passage.

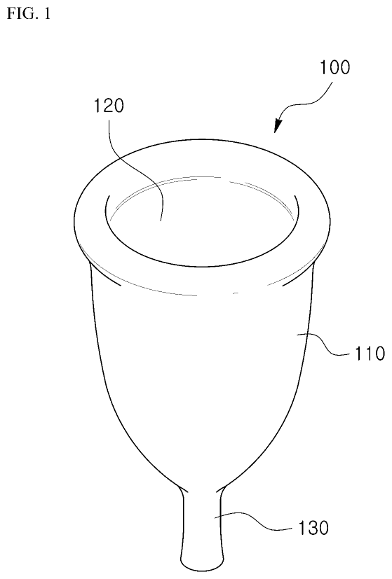

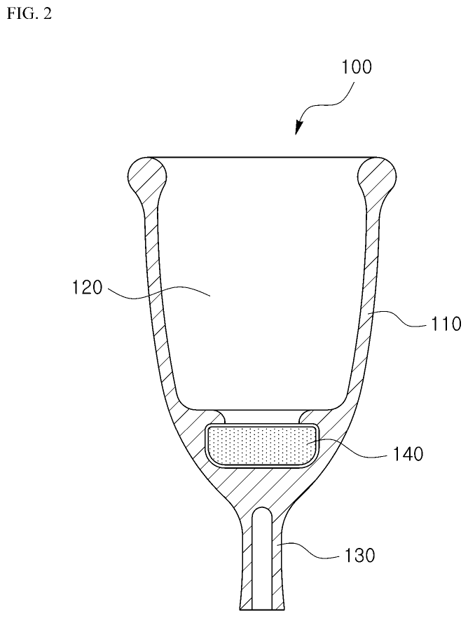

FIG. 1 is a perspective view illustrating a menstrual cup according to one embodiment of the present invention, and FIG. 2 is a cross-sectional view illustrating a menstrual cup according to one embodiment of the present invention.

Referring to FIG. 1, a menstrual cup 100 according to one embodiment of the present invention includes a body part 110 and a handle 130.

The body part 110 of the menstrual cup 100 according to the present embodiment is formed in a cup shape, and a storage part 120 is formed in the body part 110 to store menstrual blood therein. In the present embodiment, a shape in which a diameter of the body part 110 increases from a lower side connected to the handle toward an upper side at which an opening is formed is illustrated. However, the present invention is not limited thereto, and the body part may be formed in various shapes in which a storage part is formed.

The storage part 120 may be formed inside the body part 110 of the menstrual cup 100 according to the present embodiment of the present invention to serve to store menstrual blood which is discharged outside the body during menstruation. A size of the storage part 120 may be formed to be varied according to a capacity to store the menstrual blood.

The handle 130 of the menstrual cup 100 according to the present embodiment may be formed to protrude from a side opposite the opening of the body part 110 and may serve to facilitate insertion of the menstrual cup 100 into the vagina or removal of the menstrual cup 100 therefrom. When a length of the handle 130 is too long, it may cause discomfort when a user acts after inserting the menstrual cup 100, whereas, when the length of the handle 130 is too long, it may be difficult to remove the menstrual cup 100. Accordingly, in consideration of the above description, the length of the handle 130 may be formed appropriately. Here, a length of the handle that the user experiences inconvenience may be different from a length of the handle required to remove the menstrual cup according to the user. Therefore, the handle may be formed to be long and cuttable so as to allow the user to directly adjust the length of the handle. Meanwhile, in the present embodiment, the handle 130 of the menstrual cup 100 has been illustrated as being formed in a shape of a hollow column. However, the present invention is not limited thereto, the shape of the handle 130 may be formed in various shapes such as an annular shape, a spherical shape, and the like. Alternatively, the handle is not necessarily formed in the menstrual cup of the present invention, it is also possible to not form the handle.

During the use, the menstrual cup 100 according to the present embodiment is inserted into the vagina by bending the opening of the body part 110. After the insertion, the folded opening opens to be brought into contact with an inner wall of the vagina, and the menstrual cup 100 is fixed due to a pressure difference between an interior and an exterior of the vagina such that the menstrual cup 100 may contain menstrual blood, which is discharged outside the body, in the storage part 120 without leakage. To this end, the menstrual cup 100 may be formed of silicone having excellent elasticity and stretchability and no toxicity. However, a material of the menstrual cup is not limited thereto. In addition to the silicone, the menstrual cup may be formed of various materials with elasticity and stretchability or a portion of the menstrual cup may be formed of a material having no elasticity and no stretchability

As described above, the menstrual blood contained in the storage part of the menstrual cup may have many biometric information such as genetic information, protein information, and the like. Thus, in one embodiment of the present invention, a cartridge blood test is provided so as to test the menstrual blood and obtain the biometric information.

Referring to FIG. 2, the menstrual cup 100 according to the present embodiment further includes a cartridge 140 which is disposed below storage part 120 and capable of testing the menstrual blood. Specifically, a space which is capable of being connected to the storage part 120 and receiving the cartridge 140 is provided at a lower side (a handle side) of the storage part 120, and the cartridge 140 is mounted in such a receiving storage space in a removable manner.

The cartridge 140 according to the present embodiment is a kind of lab-on-a-chip. The cartridge 140 may have an internal structure which is capable of capturing and testing the menstrual blood contained in the storage part 120. The test result performed in the cartridge 140 may be checked by removing the cartridge 140 from the menstrual cup 100 and connecting the cartridge 140 to a separately provided cartridge reader.

Meanwhile, the menstrual cup 100, which is mounted in the vagina, has a cross-sectional diameter ranging from about 3 cm to about 5 according to a capacity of a product. Therefore, the cartridge 140 according to one embodiment of the present invention is formed by stacking a plurality of sections so as to be mounted on the menstrual cup by minimizing a cross-sectional area of the cartridge 140. The cartridge 140 has a structure in which blood in each section is movable using gravity, a capillary action, and the like.

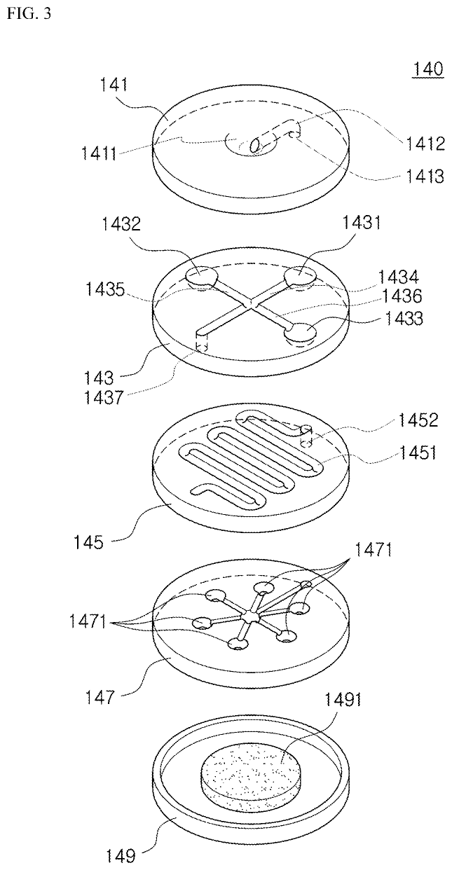

FIG. 3 is an exemplary diagram illustrating an internal structure of a cartridge provided in the menstrual cup according to one embodiment of the present invention. Referring to FIG. 3, the cartridge 140 according to one embodiment of the present invention may be constituted such that menstrual blood introduced from the outside of the cartridge 140 reacts with a reaction solution while passing through a plurality of stacked sections and a target material detected through the reaction is stored.

The cartridge 140 according to the present embodiment is provided for a gene analysis. As shown in FIG. 3, the cartridge 140 may include a blood input section 141, a reaction section 143, a separation analysis section 145, a retention section 147, and a device connector 149.

The blood input section 141 of the cartridge 140 according to one embodiment of the present invention is connected to the storage part 120 of the menstrual cup 100 and serves as an inlet through which blood is introduced into the cartridge 140. To this end, a groove 1411 may be formed at a center of the blood input section 141, and a fine fluidic tube 1412 may be connected to the central groove 1411, and one end of the fine fluidic tube 1412 may be connected to a first connection hole 1413 to move the menstrual blood downward.

According to one embodiment of the present invention, the blood input section 141 may include a filter (not shown) for preventing an inflow of foreign materials other than the blood. The menstrual blood contains foreign materials such as endometrial cells, menstrual blood masses, and other secretions, and the like in addition to the blood. When the foreign materials are introduced into the cartridge 140 together with the blood, a flow path such as a fine fluidic tube through which the blood moves may be blocked and may also affect a test result. Accordingly, in one embodiment of the present invention, the filter may be provided in the blood input section 141 to block an inflow of foreign materials.

In the present embodiment, the filter may be disposed on an upper end of the blood input section 141. The filter may be formed in a structure including one or more fine hole by perforation. However, a position and the structure of the filter are not limited thereto, and the position and the structure thereof may be variously changed.

The reaction section 143 may be disposed below the blood input section 141 of the cartridge 140 according to the present embodiment. The reaction section 143 is connected to the blood input section 141 through the first connection hole 1413. According to the purpose of analysis, the reaction section 143 may perform a function of mixing the blood introduced from the blood input section 141 with a reaction solution such that the blood reacts the reaction solution.

To this end, the reaction section 143 includes a blood storage groove 1431 in which the blood moving from the blood input section 141 are collected, a reaction solution storage groove 1432 in which the reaction solution is stored, and a buffer solution groove 1433 in which a buffer solution is stored. A second channel 1435 and a third channel 1436, which are connected to the blood storage groove 1431 and connected to a first channel 1434, the reaction solution storage groove 1432, and the buffer solution groove 1433 through which the blood is movable, are formed in the reaction section 143. Further, the second channel 1435 and the third channel 1436 may be connected to the first channel 1434, and one end of the first channel 1434 may be connected to a second connection hole 1437. The reaction solution may be a material which is capable of binding to a target material, which will be detected, e.g., a specific deoxyribonucleic acid (DNA) or ribonucleic acid (RNA). The buffer solution may perform a function of maintaining a pH at a predetermined value when the reaction solution reacts the blood.

With the above configuration, the blood introduced into the blood storage groove 1431 through the fine fluidic tube 1412 in the blood input section 141 moves along the first channel 1434 and is mixed with the reaction solution and the buffer solution, which are introduced from the second channel 1435 and the third channel 1436 such that the blood reacts the reaction solution and the buffer solution.

The separation analysis section 145 of the cartridge 140 according to the present embodiment may be disposed below the reaction section 143 and may perform a function of separating a target material from the blood mixed with the reaction solution. A fourth channel 1451 is formed in the separation analysis section 145, and a third connection hole 1452 is connected to one end of the fourth channel 1451 so that the blood mixed with the reaction solution may be introduced into the fourth channel 1451 through the second connection hole 1437 connected to one end of the first channel 1434 of the reaction section 143 to flow along the fourth channel 1451. In order to separate the target material from the blood mixed with the reaction solution, an electric field or surface tension may be used in the separation analysis section 145, or a separate micro pump may be used therein. However, in addition to the above-described method, other known method of separating the target material may be applied.

The retention section 147 of the cartridge 140 according to the present embodiment is disposed below the separation analysis section 145 and performs a function of storing the target material which is detected by passing through the reaction section 143 and the separation analysis section 145. Specifically, at least one well 1471 is formed in the retention section 147. The target material, which is separated by passing through the fourth channel 1451 of the separation analysis section 145, may be introduced through the third connection hole 1452 to be stored in each well 1471. In this case, when a plurality of target materials, which will be detected through the cartridge 140, are present, a plurality of wells 1471 may be formed in different forms according to the target materials which will be detected.

Meanwhile, in the present embodiment, the separation analysis section 145 has been illustrated as being disposed as a separate section between the reaction section 143 and the retention section 147, but the separation analysis section 145 can be removed. In this case, the function of the separation analysis section may be changed to perform in the reaction section 143 or the retention section 147 which will be described below.

According to one embodiment of the present invention, the device connector 149 is disposed below the retention section 147. When the cartridge 140 is removed from the device and then installed in a cartridge reader so as to check the test result, the device connector 149 according to the present embodiment may perform a function of connecting the cartridge 140 to the cartridge reader. Meanwhile, as shown in the drawing, a detector 1491 which is capable of detecting a target material may be provided in the device connector 149. Though the detector 1491, the target material stored in the well 1471 of the retention section 147 may be detected.

As described above, it is possible to check the test result by directly connecting the cartridge to the cartridge reader through the device connector 149. Alternatively, it is also possible to check the test result on the cartridge. For example, the cartridge 140 may further include a wireless communication module which is capable of transmitting data to the cartridge reader or a user terminal thereby checking the test result by transmitting analysis information to the cartridge reader or the user terminal.

According to one embodiment of the present invention, a hydrophobic material may be coated on a flow path such as a flow tube or a channel which is formed inside the cartridge 140. Owing to the coating of the hydrophobic material, a frictional force which is generated when the blood and the like flow in a fine flow path, is minimized such that a smooth flow may be achieved.

Meanwhile, although the cartridge 140 has been illustrated in a cylindrical shape in the present embodiment, a shape of the cartridge may be changed according to a shape of the cartridge receiving part inside the menstrual cup and the internal structure of the menstrual cup. For example, when the cartridge receiving part is present in the menstrual cup, in order to facilitate mounting, removal, and fixing of the cartridge, the shape may be changed to form a predetermined curvature on an outer circumferential surface of the cartridge. Each section constituting the cartridge may be formed of metal, silicone, or thermo-plastic, but the material thereof may also be variously changed.

As described above, the cartridge 140 according to one embodiment of the present invention is formed by stacking a plurality of sections for a blood test and allows the blood to move between each section using gravity, a capillary action, and the like such that the cartridge 140 is mounted on the menstrual cup and thus the blood test may be performed. Although the cartridge 140 has been illustrated as being mounted on the menstrual cup and used in one embodiment of the present invention, the cartridge 140 may be used in not only the menstrual cup but also other small device having a narrow cross-sectional area requiring a blood test.

As described above, according to one embodiment of the present invention, the menstrual cup is provided with the cartridge for blood test, and the cartridge is mounted in a form which is removable from the menstrual cup such that the test result may be checked and the cartridge may be replaced according to the need of a user. To this end, it is possible to variously modify the structure of the menstrual cup shown in FIGS. 1 and 2. Hereinafter, various structures of the menstrual cup for mounting and removing the cartridge and a method of mounting and removing the same will be described in detail with reference to the accompanying drawings. For the ease of explanation, modified embodiments of the embodiment shown in FIGS. 1 and 2 will be referred to as a first embodiment, a second embodiment, and the like.

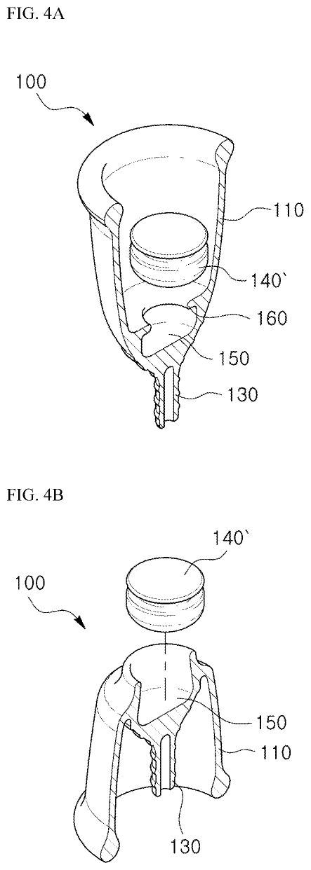

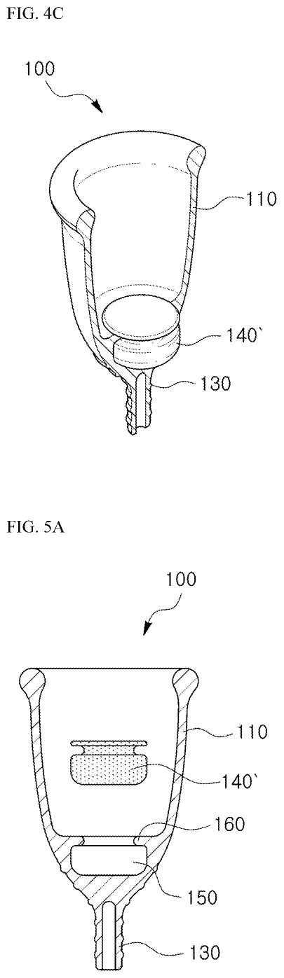

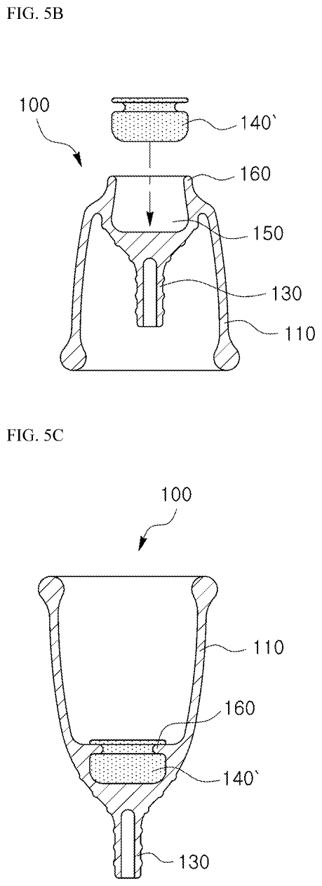

FIGS. 4A to 4C and FIGS. 5A to 5C are diagrams illustrating a case in which a cartridge is mounted in a menstrual cup according to a first embodiment of the present invention.

A menstrual cup 100′ according to a first embodiment of the present invention has a structure substantially the same as the menstrual cup 100 shown in FIG. 2. That is, the menstrual cup 100′ includes a body part 110 and a handle 130, a storage part 120 is formed inside the body part 110, and a cartridge receiving part 150, which is a space for storing a cartridge 140′, is formed below the storage part 120. Further, a hook 160 protruding inward is formed between the storage part 120 and the cartridge receiving part 150.

The present embodiment is different from the embodiment shown in FIG. 2 in that, in order to more stably fix the cartridge 140′ to the cartridge receiving part 150, a groove corresponding to the hook 160 is formed in a circumferential direction at a position adjacent to one end of the cartridge 140′.

To describe a process of mounting the cartridge 140′ in the menstrual cup 100′ according to the first embodiment of the present invention with reference to FIGS. 4A to 4C and FIGS. 5A to 5C, the menstrual cup 100′ having the hook 160 formed between the storage part 120 and the cartridge receiving part 150 and the cartridge 140′ are prepared first (see FIGS. 4A and 5A), and the body part 110 of the menstrual cup 100′ is folded inside out toward the handle 130 based on the hook 160 between the storage part 120 and the cartridge receiving part 150 (see FIGS. 4B and 5B). As described above, when the body part 110 is folded inside out, as shown in the drawings, an upper opening of the cartridge receiving part 150 is opened while the hook 160 is pushed upward (i.e., toward an original opening side of the body part 110). Thus, the cartridge 140′ may be easily inserted into the cartridge receiving part 150. When the cartridge 140′ is inserted into the cartridge receiving part 150 and then the hook 160 is hooked to the groove formed in the cartridge 140′ while the body part 110 is folded back to its original position, the cartridge 140′ may be fixedly mounted (see FIGS. 4C and 5C).

As described above, in the first embodiment of the present invention, the cartridge 140′ may be easily inserted into and fixed to the cartridge receiving part 150 having the hook 160 formed at an inlet thereof using a characteristic of the menstrual cup 100′ having excellent stretchability. According to the present embodiment, even when the cartridge 140′ is removed, the cartridge 140′ may be easily removed by folding the body part 110 inside out as in the case of mounting the cartridge 140′. As described above, the cartridge 140′ may be mounted on and removed from the menstrual cup 100′ according to the present embodiment with a simplified structure as well as may be reused because of not using a separate adhesive.

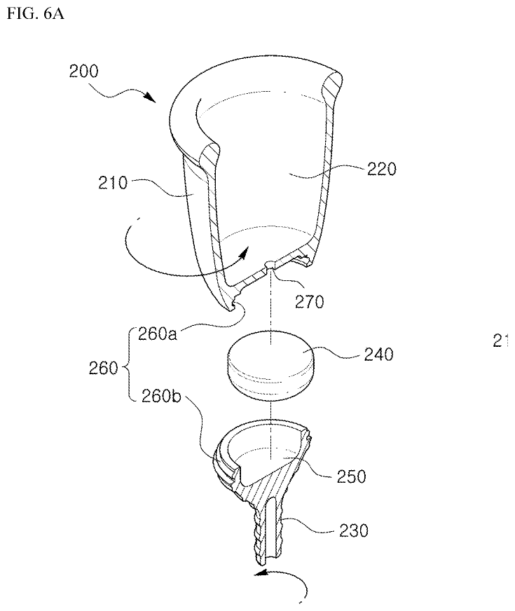

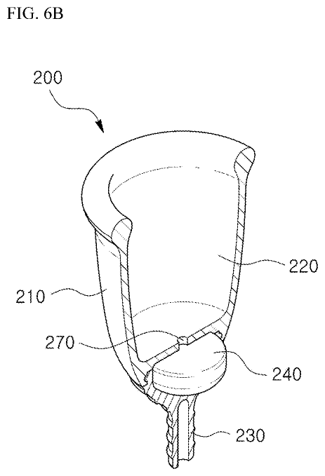

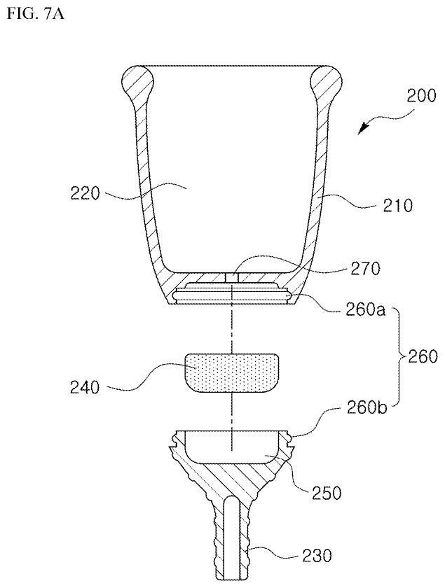

FIGS. 6A to 6B and FIGS. 7A to 7B are diagrams illustrating a case in which a cartridge is mounted in a menstrual cup according to a second embodiment of the present invention. As in the first embodiment, a menstrual cup 200 includes a body part 210 and a handle 230, a storage part 220 is formed inside the body part 210, and a cartridge receiving part 250 for storing a cartridge 240 is formed below the storage part 220.

The menstrual cup 200 according to the present embodiment is different from that of the above embodiment in that the menstrual cup 200 has a structure in which the body part 210 may be separated from and coupled to the handle 230. Specifically, in the menstrual cup 200 according to the present embodiment, a screw portion 260 is formed on each of the body part 210 and the handle 230, i.e., a female thread 260a and a male thread 260b are respectively formed on a lower end of the body part 210 and an upper end of the handle 230 such that the body part 210 is constituted to be couplable to the handle 230 due to screw engagement of the female thread 260a and the male thread 260b.

To describe a process of mounting the cartridge 240 in the menstrual cup 200 according to the second embodiment of the present invention with reference to FIGS. 6A to 6B and FIG. 7A to 7B, the body parts 210, the handle 230, and the cartridge 240, which are separated from each other, are prepared first, and the cartridge 240 is inserted into the cartridge receiving part 250 formed on the handle 230 (see FIGS. 6A and 7A). Subsequently, the female thread 260a and the male thread 260b formed on the body part 210 and the handle 230 are aligned and rotated in opposite directions to be coupled.

As shown in FIGS. 6B and 7B, through the above process, the cartridge 240 may be stably mounted on and fixed to the cartridge receiving part 250 formed below the storage part 220. Meanwhile, a blood inlet 270 connected to the storage part 220 may be formed above the cartridge receiving part 250. Thus, in a state in which the cartridge 240 is mounted, the cartridge 240 may receive menstrual blood from the storage part 220 through the blood inlet 270 and perform a test of the menstrual blood.

The cartridge 240 may be removed by performing the above process in a reverse order. That is, the cartridge 240 may be removed from the cartridge receiving part 250 which is opened by releasing the screw engagement between the body part 210 and the handle 230.

As described above, in the second embodiment of the present invention, the body part 210 and the handle 230 of the menstrual cup 200 are separably formed and the screw structure is applied to the body part 210 and the handle 230 such that the cartridge 240 may be easily mounted and removed. According to the present embodiment, as in the above-described embodiments, the structure is simplified and a separate adhesive is not such that the menstrual cup may be reused.

Meanwhile, although the body part 210 and handle 230 have been described as being separated from and coupled to each other in the present embodiment, it may be understood that the body part has a structure of being separated from and coupled to a first body part and a second body part according to a position at which the separation and the coupling are performed, and the handle is formed to protrude from the second body part. In this case, a female thread and a male thread are respectively formed on the first body part and the second body part and thus screw engagement may be performed.

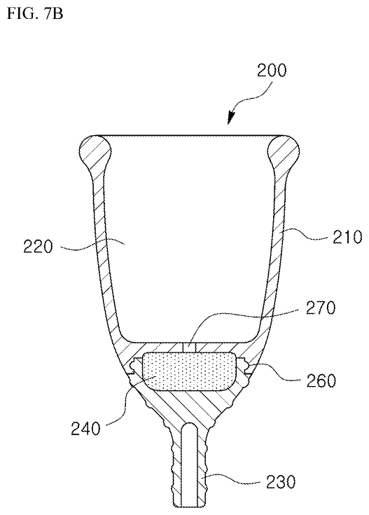

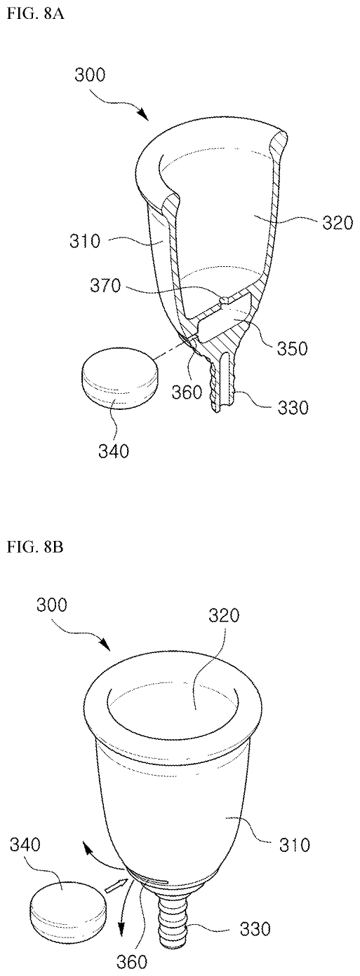

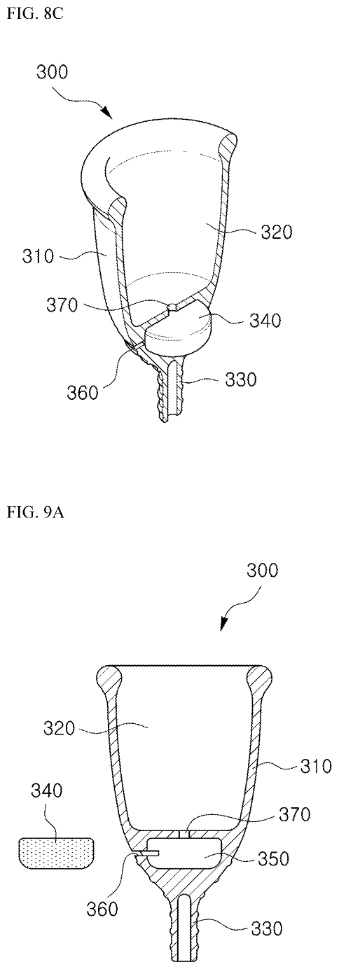

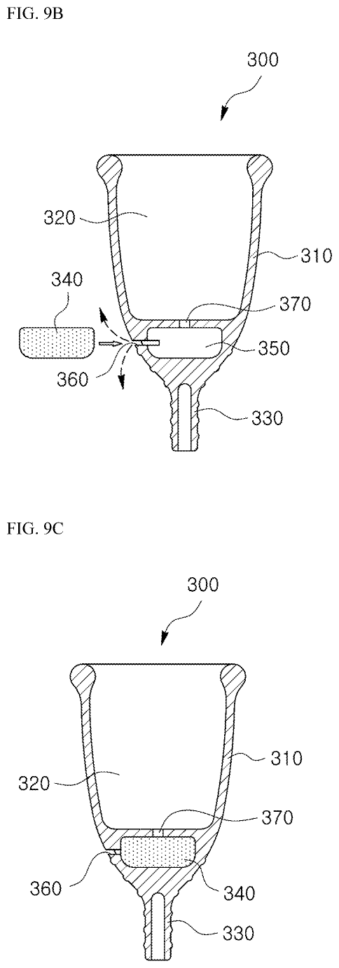

FIGS. 8A to 8C and FIGS. 9A to 9C are diagrams illustrating a case in which a cartridge is mounted in a menstrual cup according to a third embodiment of the present invention. As in the above embodiments, a menstrual cup 300 according to a third embodiment of the present invention includes a body part 310 and a handle 330, a storage part 320 is formed inside the body part 310, and a cartridge receiving part 350 is formed below the storage part 320. Further, a blood inlet 370 is formed between the storage part 320 and the cartridge receiving part 350.

A cut-out portion 360 is further formed on one side of the menstrual cup 300 according to the present embodiment. The cut-out portion 360 serves as a passage connected to the cartridge receiving part 350 from the outside of the menstrual cup 300. Through the cut-out portion 360, the cartridge 340 may be easily inserted into the cartridge receiving part 350.

To describe a process of mounting the cartridge 340 in the menstrual cup 300 according to the third embodiment of the present invention with reference to FIGS. 8A to 8C and FIGS. 9A to 9C, the menstrual cup 300 having one side at which the cut-out portion 360 is formed and the cartridge 340 are prepared first (see FIGS. 8A and 9A). Thereafter, the cut-out portion 360 is vertically opened to enlarge a passage with the cartridge receiving part 350, and the cartridge 340 is inserted into the cartridge receiving part 350 through the enlarged passage (see FIGS. 8B and 9B). Consequently, as shown in FIGS. 8C and 9C, the cartridge 340 may be mounted on the cartridge receiving part 350.

In the present embodiment, the cartridge 340 may be easily and stably inserted and fixed to the cartridge receiving part 350 through the cut-out portion 360 by utilizing a characteristic of the menstrual cup 300 having excellent stretchability, and the mounted cartridge 340 may receive menstrual blood from the storage part 320 through the blood inlet 370 and perform a test of the menstrual blood.

Similarly, even when the cartridge 340 is removed, the cartridge 340 may be pulled out through the passage enlarged by vertically opening the cut-out portion 360. The cut-out portion 360 may be formed to have a size which the cartridge 340 may enter and exit when the cut-out portion 360 is maximally opened to extend. This may vary according to stretchability of the menstrual cup 300, i.e., a material of the menstrual cup 300.

As described above, in the third embodiment of the present invention, the cut-out portion 360 is formed on one side of the menstrual cup 300 and the cartridge 340 easily enters and exits using the stretchability of the menstrual cup 300 such that the mounting and removal of the cartridge 340 may be easily performed. Therefore, as in the above embodiments, the present embodiment also enables the reuse of the menstrual cup while having a simplified structure.

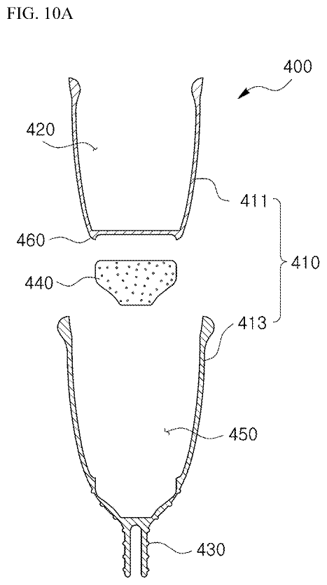

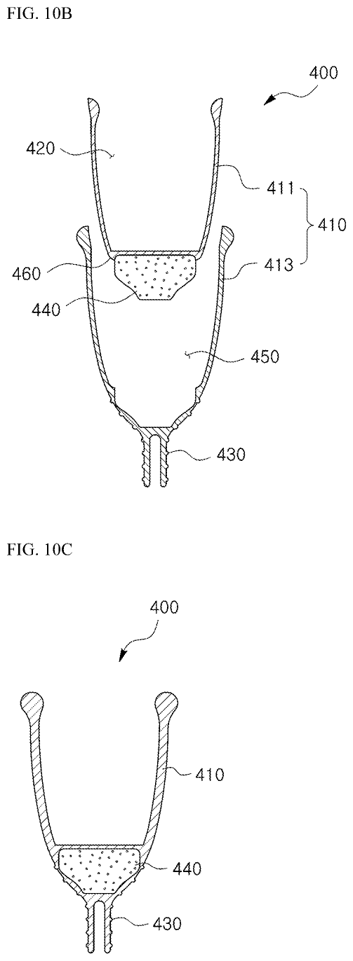

FIGS. 10A to 10C are diagrams illustrating a case in which a cartridge is mounted in a menstrual cup according to a fourth embodiment of the present invention. Referring to FIG. 10A to 10C, a menstrual cup 400 according to the present embodiment includes a body part 410 and a handle 430, a storage part 420 for containing menstrual blood therein is formed inside the body part 410, and a cartridge receiving part 450 for storing a cartridge 440 is formed below the storage part 420.

The present embodiment is similar to the above embodiments in that the cartridge receiving part 450 is formed so that the cartridge 440 may be removably mounted in the menstrual cup 400, and the cartridge receiving part 450 is located below the storage part 420. However, the present embodiment is different from the above embodiments in that the body part 410 of the menstrual cup 400 is divided into an inner cup 411 and an outer cup 413.

Specifically, in the menstrual cup 400 according to the present embodiment, when the inner cup 411 and the outer cup 413 of the body part 410 are coupled, a space for storing the cartridge 440, i.e., the cartridge receiving part 450, is formed between the inner cup 411 and the outer cup 413 so that the menstrual cup 400 has a structure in which mounting and removal of the cartridge 440 is possible through coupling and separation of the inner cup 411 and the outer cup 413.

To describe a process of mounting the cartridge 440 in the menstrual cup 400 according to the present embodiment in detail, the menstrual cup 400 having a state in which the inner cup 411 is separated from the outer cup 413 is prepared first (see FIG. 10A). Then, the cartridge 440 is mounted in the inner cup 411 (see FIG. 10B). In the present embodiment, it has been illustrated that a step bump 460 was formed on an outer circumference of a lower end of the inner cup 411 so that the cartridge 440 was fitted into a groove formed inside the lower end thereof. However, a method of mounting the cartridge 440 on the inner cup 411 is not necessarily limited thereto.

Subsequently, the inner cup 411 is coupled to the outer cup 413 such that the cartridge 440 is located in the cartridge receiving part 450 which is a space formed in the inner cup 411 and the outer cup 413 (se FIG. 10C). The coupling of the inner cup 411 and the outer cup 413 may be made by an adhesive material such as silicone adhesive or may be made through a mechanical engagement structure such as a zipper on an upper end.

As described above, in the present embodiment, since the inner cup 411 and the outer cup 413 of the body part 410 are formed to be separated from and coupled to each other, a user may easily mount or remove a required cartridge 440. Meanwhile, in the present embodiment, the coupling of the inner cup 411 and the outer cup 413 has been described as being performed in a state in which the cartridge 440 was mounted on the inner cup 411, this is merely illustrative. The cartridge 440 may be mounted on either the inner cup 411 and the outer cup 413, and, alternatively, in a state in which the cartridge 440 is not physically mounted (coupled) to the inner cup 411 and the outer cup 413, the coupling of the inner cup 411 and the outer cup 413 may be performed by interposing the cartridge 440 between the inner cup 411 and the outer cup 413.

While the present invention has been described with reference to specific items such as particular components, exemplary embodiments, and drawings, these embodiments and drawings are merely provided to help understanding the present invention, and the present invention is not limited to these embodiments, and those skilled in the art to which the present invention pertains can variously alter and modify from the description of the present invention.

For example, in the first embodiment of the present invention, the method of inserting the cartridge after folding the body part inside out in a direction of the handle has been illustrated. However, it is possible to mount the cartridge by directly inserting the cartridge into the hook in a state in which the body part is folded inside out. Further, in the second embodiment of the present invention, the body part and the handle, which are separated by interposing the cartridge between the body part and the handle, have been described as being coupled in a screw structure. However, the coupling method of the body part and the handle can be changed in various forms such as a fitting method in which one member of the body part and the handle is inserted into and fixed in the other member, or a method of forming a hooking bump on one member and forming a hooking groove on the other member so that the hooking bump and the hooking groove are coupled.

Therefore, the spirit of the present invention should not be limited to the above-described embodiments, and it should be construed that the appended claims as well as all equivalents or equivalent modifications of the appended claims will fall within the scope of the present invention.

What is claimed is:

1. A menstrual cup having a blood test cartridge, comprising:
a body part having an opening formed at one side of the body part, a storage part configured to receive menstrual blood therein, and a cartridge receiving part connected to the storage part at a side opposite the opening; and
a cartridge for blood test removably mounted in the cartridge receiving part.

2. The menstrual cup of claim 1, further comprising:
a handle formed to protrude from the side opposite the opening of the body part.

3. The menstrual cup of claim 1, wherein a hook configured to mount and fix the cartridge is formed between the cartridge receiving part and the storage part.

4. The menstrual cup of claim 3, wherein a groove is formed in the cartridge at a position corresponding to the hook such that the hook is hooked when the cartridge is received in the cartridge receiving part.

5. The menstrual cup of claim 1, wherein the body part comprises a first body part and a second body part which can be separated and coupled by interposing the cartridge receiving part therebetween.

6. The menstrual cup of claim 5, wherein a screw portion is formed in each of the first body part and the second body part for mutual coupling.

7. The menstrual cup of claim 5, wherein a handle is formed to protrude from the second body part.

8. The menstrual cup of claim 1, wherein:
a cut-out portion configured to connect the cartridge receiving part to the outside is formed outward the cartridge receiving part; and
the cartridge is insertable into the cartridge receiving part through the cut-out portion.

9. The menstrual cup of claim 1, wherein:
the body part comprises an inner cup and an outer cup which can be coupled to and separated from each other; and
when the inner cup is coupled to the outer cup, the cartridge receiving part is formed between the inner cup and the outer cup.

10. The menstrual cup of claim 9, wherein:
the cartridge is mounted in the inner cup when the inner cup is separated from the outer cup and
the cartridge is located in the cartridge receiving part formed between the inner cup and the outer cup when the inner cup is coupled to the outer cup.

11. The menstrual cup of claim 1, wherein the cartridge is capable of performing genetic analysis or protein analysis of blood or detecting a disease.

12. The menstrual cup of claim 1, wherein the cartridge has a structure in which a plurality of sections for blood test are stacked.

13. The menstrual cup of claim 1, wherein the cartridge comprises a wireless communication module.

14. The menstrual cup of claim 1, wherein the cartridge is configured to transmit a test result to a user terminal.
